(19) United States
(12) Patent Application Publication
(10) Pub. No.: US 2026/0345225 A1
(43) Pub. Date: Nov. 5, 2026

(54) ELECTROSURGICAL TOOL AND METHOD FOR PRODUCING SAME

(71) Applicant: KARL STORZ SE & Co. KG, Tuttlingen (DE)

(72) Inventors: Thomas HINDING, Tuttlingen (DE); Sebastian WAGNER, Tuttlingen (DE); Sebastian WENZLER, Tuttlingen (DE)

(21) Appl. No.: 19/118,856

(22) PCT Filed: Oct. 4, 2023
(86) PCT No.: PCT/EP2023/077429

§ 371 Date: Apr. 7, 2025

(30) Foreign Application Priority Data
Oct. 5, 2022 (DE) 10 2022 125 714.2

(51) Int. Cl. A61B 18/14; A61B 18/00

(52) U.S. Cl. CPC A61B 18/1445; A61B 2018/00595; A61B 2018/00601

(57) ABSTRACT

An electrosurgical tool (40), for an electrosurgical instrument (10), includes a first jaw part (52), a second jaw part (54), and a flexible conductor component (60). The flexible conductor component (60) includes an electrically insulating substrate (64) and a plurality of first electrode surfaces (82) on a first surface region (82) of the flexible conductor component (60). At least one first part area (72) of the flexible conductor component (60) is mechanically connected to the first jaw part (52). The first surface region (82) forms at least one part of a first gripping surface (56) on the first jaw part (52) in the first part area (72) of the flexible conductor component (60) that includes the plurality of first electrode surfaces (86).

### CROSS REFERENCE TO RELATED APPLICATIONS

This application is a United States National Phase Application of International Application PCT/EP2023/077429, filed Oct. 4, 2023, and claims the benefit of priority under 35 U.S.C. § 119 of German Application 10 2022 125 714.2, filed Oct. 5, 2022, the entire contents of which are incorporated herein by reference.

### TECHNICAL FIELD

The present invention is directed to an electrosurgical tool, an electrosurgical instrument having an electrosurgical tool, and a method of manufacturing an electrosurgical tool.

### BACKGROUND

In electrosurgery, tissue is heated electrothermally in order to denature, coagulate, cauterize, vaporize, ablate or sever it. Electrosurgical instruments and the electrosurgical tools at the distal ends of electrosurgical instruments continue to be miniaturized and undergo further improvements. This includes the use of a higher number of electrode surfaces in order to be able to control current flows more precisely.

EP 1 632 192 A1 describes an instrument for closing vessels, which has two gripping jaws that can be moved relative to one another. Each gripping jaw comprises a pair of electrically conductive, spaced vessel closure surfaces extending along the length of the gripping jaw. Each pair of vessel occlusion surfaces is connected to a source of electrosurgical energy. The gripping jaws can be configured as a mirror image or not.

US 2007/0156139 A1 (also published as U.S. Pat. No. 7,776,036 B2) describes a bipolar electrosurgical forceps with a pair of opposing jaws with tissue-gripping surfaces. A plurality of ring-like electrodes are provided on one of the surfaces gripping the tissue, while a plurality of post electrodes are arranged on the other surface gripping the tissue.

US 2010/274244 A1 (also published as U.S. Pat. No. 8,277,446 A1) describes an instrument for electrosurgical closure and cutting of tissue. The instrument comprises a pair of jaw parts with opposing tissue gripping surfaces on which electrodes are arranged in a row and insulated from one another. The instrument further comprises a control for supplying electrical energy to a particular electrode while other electrodes are in an inactive state. A sensor array communicating with the control unit detects a characteristic of the tissue that indicates a complete electrosurgical treatment. The sensor array can include an impedance sensor.

US 2016/0287316 A1 (also published as U.S. Pat. No. 10,813,684 B2) describes the control of electrosurgical closure and severing of tissue. Electrodes on gripping surfaces on jaw parts are divided into zones. Each zone can include a monitoring device to measure the impedance in the zone.

US 2016/0302854 A1 (also published as U.S. Pat. No. 9,717,550 B2) describes an electrosurgical forceps with jaw parts whose gripping surfaces or closing plates each consist of several segments that are electrically insulated from each other by insulating components. The segments may be preselected or dynamically selected as components of one or more electrical circuits that provide electrosurgical energy to tissue located between the jaw parts. For example, a first bipolar circuit may comprise outer segments, a second bipolar circuit may comprise middle segments, and a third bipolar circuit may comprise inner segments, wherein the first, second and third circuits may be activated independently of one another to deliver electrosurgical energy to the tissue. A generator with a controller, a sensor module and a multiplexer measures the impedance of tissue positioned between two selected segments, and based on the measured values, the generator forms one or more bipolar circuits between selected segments (paragraph [0049]). Each closing plate is connected to the associated jaw part housing by a plate holder, which can be configured as part of the closing plate or as part of the jaw part housing or as an independent component. The plate holder may comprise a circuit, circuit board or connections connecting the segments to the electrical power source.

EP 2 149 342 A1 describes a multiphase electrosurgical system with an array of electrodes. A multiphase high-frequency generator is configured to generate a plurality of independent high-frequency signals whose phase, amplitude and/or frequency can be varied independently. The high voltage signals may have substantially sinusoidal or non-sinusoidal waveforms, and each independent signal may be applied to a corresponding electrode of the surgical instrument. A sensor module can record biometric parameters such as tissue impedance “corresponding to the application of the electrosurgical waveforms to the tissue”. Depending on the biometric parameter, a controller can adjust the phase, amplitude and/or frequency, wherein the formation of vapor bubbles is mentioned (ibid.). The figures show parallel needle-shaped electrodes arranged at the corners of regular polygons or forming the corners of rows of triangles.

### SUMMARY

One object of the invention to of the present invention is to provide an improved electrosurgical tool and an improved method for manufacturing an electrosurgical tool.

This object is achieved by features according to the invention.

Further embodiments are presented with this disclosure.

An electrosurgical tool for an electrosurgical instrument comprises a first jaw part, a second jaw part and a flexible conductor component having an electrically insulating substrate and a plurality of first electrode surfaces on a first surface area (first surface region) of the flexible conductor component, wherein at least a first part area of the flexible conductor component is mechanically connected to the first jaw part, wherein the first surface area of the flexible conductor component with the plurality of first electrode surfaces forms at least part of a first gripping surface on the first jaw part.

The flexible conductor component is particularly flat, i.e. thin and substantially two-dimensional. This means that its surface is substantially formed by two surface areas facing away from one another and substantially parallel, with the distance between the surface areas being much smaller than the linear dimensions of the surface areas.

A surface area of the first jaw part facing the second jaw part forms the first gripping surface of the first jaw part. A surface area of the second jaw part facing the first jaw part forms the second gripping surface of the second jaw part. Both gripping surfaces are configured to correspond in particular, so that both gripping surfaces can lie flat or substantially flat against one another. One or both gripping surfaces can be structured to prevent the gripped tissue from slipping away, thus enabling more secure gripping, or to improve the electrosurgical closure of vessels or other tissue. One or both gripping surfaces have a structure or profiling, for example similar to a waffle or a waffle iron. The structure or profiling has the shape of a rectangular, oblique-angled or hexagonal grid, for example. The structure or profiling can repeat periodically or quasiperiodically or non-periodically in one or two directions. Both gripping surfaces can then touch one another in many small, regularly or irregularly arranged, but in particular substantially constant density, point-like or more extended surfaces or in a grid of non-intersecting or intersecting lines. In these cases, too, one gripping surface substantially lies flat against the other gripping surface.

Alternatively, both gripping surfaces are shaped in such a manner that both gripping surfaces are substantially parallel to one another at a predetermined distance. This also does not exclude a profile or structuring of one or both gripping surfaces as described above.

In the case of a gripping surface with profile or structuring, the flexible conductor component and a surface area of an underlying mechanical structural component of the jaw part are in particular conformal, i.e. have corresponding geometric shapes, so that the flexible conductor component lies over the entire surface or substantially over the entire surface of the surface area of the underlying mechanical structural component of the jaw part.

A profile or structuring of a flexible conductor component does not necessarily require a non-zero Gaussian curvature. For example, the flexible conductor component can be folded or pleated—similar to origami.

Alternatively, the flexible conductor component can have a non-zero Gaussian curvature locally or over a large area. A flexible conductor component can already be originally manufactured with this Gaussian curvature or even with the specific profile or structuring. Alternatively, the flexible conductor component originally produced flat or with disappearing Gaussian curvature can be given the profile or structure by (optionally thermally assisted) plastic deformation, for example embossing.

The electrode surfaces of the plurality of electrode surfaces are exposed at the first gripping surface at the first jaw part. This means that each individual electrode surface of the plurality of electrode surfaces between the gripping surfaces of the jaw parts can directly mechanically contact held or crushed tissue and form a conductive contact with them.

In particular, the electrode surfaces of the plurality of electrode surfaces are electrically insulated from one another and can be connected or are connected independently of one another to an electrical power source and/or an electrical measuring device for measuring an impedance and/or another electrical property or parameter. Alternatively, several first electrode surfaces can be electrically connected in parallel within one or more groups.

Measuring the impedances and/or other electrical properties or characteristics between different pairs of electrode surfaces can enable spatially differentiated detection of the electrical properties of tissue between the jaw parts. Ideally, measuring the impedances and/or other electrical properties or characteristics between very many different pairs of electrode surfaces can allow approximate tomographic detection of the electrical properties of tissue between the jaw parts. Likewise, the multiple electrode surfaces on each gripping surface can enable a spatially differentiated current supply and thus also a spatially differentiated electrosurgical effect, in particular heating and cauterization of tissue. For example, depending on the spatially differentiated condition of the tissue, the voltage, current and/or applied power can be limited in a locally differentiated manner in order to prevent the tissue from overheating. The properties of the tissue can be recorded first and then the tissue energized or, alternatively, the recording and energization can take place alternately or intermittently or partially or completely simultaneously.

The flexible conductor component can be permanently mechanically connected to the first jaw part. This means that it cannot be non-destructively detached from the first jaw part without using a screwdriver, wrench or other tool, or cannot be non-destructively detached from the first jaw part even when a screwdriver, wrench or other tool is used. A permanent mechanical connection can be achieved by gluing, soldering, welding or overmolding, for example. It would not be non-destructive to separate the flexible conductor component from the first jaw part by cutting or sawing or by destroying an adhesive or welded connection.

Alternatively, the flexible conductor component can be mechanically connected to the first jaw part in such a way that it can be separated from the first jaw part non-destructively without using a screwdriver, wrench or other tool. For example, the flexible conductor component has a pocket that receives the first jaw part or a portion of the first jaw part in the intended use of the electrosurgical tool. Alternatively or additionally, the flexible conductor component can be detachably mechanically connected to the first jaw part, for example by means of a snap-in connection, such as one or more press studs. Alternatively or additionally, the flexible conductor component can be detachably mechanically connected to the first jaw part, for example by a dovetail guide or in another form-fit manner. Alternatively or additionally, the flexible conductor component can be held on the first jaw part by magnetic or electrostatic forces, for example.

The flexible conductor component is in particular a flexible printed circuit board or a flex circuit board, as used in many electrical or electronic devices. Such a flexible printed circuit board often comprises one or more films made of polyimide or another plastic. Electrically conductive tracks (conductor tracks) can be arranged on one or both surfaces of the film. In the case of several polyimide films, electrically conductive tracks can be arranged between them, which can thus be electrically insulated from the environment.

Alternatively, the flexible conductor component can be manufactured partially or completely as a MID (Molded Interconnect Device or Mechatronic Integrated Device).

Flexible conductor components, in particular flexible printed circuit boards or flex circuit boards, are available in a wide variety and at low cost. Both the thickness of the flexible conductor component and the structure sizes of conductor tracks on and in a flexible conductor component, their geometric shape and arrangement are freely selectable within wide limits and can be adapted to the requirements of the application. Flexible conductor components or at least their surfaces can be made of medically safe materials. Insulating distances on and in flexible conductor components can be dimensioned for high voltages. The current carrying capacity of conductor tracks in flexible conductor components can be adjusted within wide limits.

The use of a flexible conductor component and the formation of electrode areas on its surface can be a cost-effective and technically and medically convincing solution. This applies both to fully reusable electrosurgical tools, which can be used again after cleaning and sterilization, and to electrosurgical tools that are intended and configured in part or in full for single use only. For example, the flexible conductor component may be intended and configured for single use and the jaw parts and other components of the electrosurgical tool for multiple use.

In an electrosurgical tool as described herein, in particular at least either the first gripping surface on the first jaw part or the second gripping surface on the second jaw part is flat or substantially flat.

An electrosurgical tool as described herein is in particular a gripping tool with several jaw parts that can be moved towards and away from one another manually or by motor.

The electrosurgical tool may have two or more jaw parts, one or more of which are movable, in particular pivotable. The electrosurgical tool can be provided and configured for gripping, holding, squeezing tissue between the jaw parts.

In an electrosurgical tool as described herein, the flexible conductor component comprises, in particular, a plurality of conductor tracks (76), each of the plurality of conductor tracks being electrically conductively connected to one of the plurality of first electrode surfaces.

The conductor tracks are formed from one or more electrically conductive materials and are electrically insulated from one another and, in particular, from the surroundings of the flexible conductor component. For this purpose, the conductor tracks can be arranged in the electrically insulating substrate or covered by an electrically insulating material, such as a lacquer.

The conductor tracks can merge into the electrode surfaces and be manufactured in one piece and/or simultaneously with them. For example, an area not covered by electrically insulating material and optionally widened at a distal end of each conductor track forms an electrode surface.

The conductor tracks are intended and configured to connect the electrode surfaces independently of one another to an electrical power source and/or to an electrical measuring device.

In particular, an electrosurgical tool as described herein comprises a joint hingedly connecting at least one of the first jaw part and the second jaw part to a distal end of a shaft or to a mechanical coupling device or to a handling device, wherein the flexible conductor component extends from the first jaw part across the joint to a location proximal to the joint.

The joint can have one or more degrees of freedom. In particular, the joint enables pivoting about a pivot axis of predetermined spatial orientation relative to one or both jaw parts.

The coupling device is provided and configured in particular for the non-destructive detachable mechanical connection of the electrosurgical tool to a corresponding coupling device at a distal end of a shaft or on a handling device, even without the use of tools.

The location proximal to the joint to which the flexible conductor component extends is, in particular, immediately proximal to the joint or at a proximal end of a shaft or in a handling device to which the electrosurgical tool is mechanically and functionally connected.

By extending from the electrode surfaces to a location proximal to the joint, the flexible conductor component with the conductor tracks on or in it can create an electrically conductive connection across the joint.

In an electrosurgical tool as described herein, the flexible conductor component extends in particular from the first jaw part through the joint or past the joint to a handling device.

The handling device can be directly mechanically connected to the electrosurgical tool. Alternatively, the handling device can be indirectly mechanically connected to the electrosurgical tool, in particular by means of a long and thin, straight or curved, rigid or flexible shaft.

Optionally, the flexible conductor component may extend proximally beyond the handling device, for example similar to a cable or within a cable to an electrical power source and/or an electrical measuring device for measuring an impedance and/or another electrical property or characteristic.

In an electrosurgical tool as described herein, the flexible conductor component extends in particular from the first jaw part via the joint to a proximal end of a shaft whose distal end is mechanically connected to the electrosurgical tool.

Similar to how in the case of many conventional minimally invasive instruments a push or pull rod or other mechanical transmission device extends to the handling device, the flexible conductor component can extend to the handling device. This applies regardless of whether the electrosurgical tool is mechanically connected to the shaft in a permanent or non-destructive detachable manner, and regardless of whether the shaft is mechanically connected to the handling device in a permanent or non-destructive detachable manner.

In particular, an electrosurgical tool as described herein comprises an electrical connector mechanically and electrically connected to a proximal end of the flexible conductor component.

The electrical connector is in particular an electrical plug connector. The electrical connector in particular is permanently mechanically connected to the flexible conductor component. The proximal end of the flexible conductor component can form part of the electrical connector. In particular, exposed proximal ends of conductor tracks of the flexible conductor component can form contacts of the electrical connector.

In an electrosurgical tool as described herein, the proximal end of the flexible conductor component is configured in particular as a plug connector for mechanical and electrical connection to a corresponding plug connector or can be mechanically and electrically connected to a plug connector.

An electrosurgical tool as described herein comprises in particular a mechanical transmission device for transmitting a manually or mechanically generated force and/or a manually or mechanically generated torque to the electrosurgical tool, for moving at least either the first jaw part or the second jaw part, wherein the flexible conductor component is integrally or mechanically rigidly connected to the mechanical transmission device.

The mechanical transmission device is provided and configured for the mechanical coupling of the electrosurgical tool with a handling device. This mechanical coupling means that a movement generated manually or mechanically on the handling device is accompanied by a corresponding movement on the electrosurgical tool. For example, the mechanical transmission device couples a component of the handling device that can be manually pivoted or rotated or displaced relative to the rest of the handling device or the proximal end of the shaft to one or more jaw parts of the electrosurgical tool such that any movement of the component relative to the rest of the handling device is accompanied by a movement of the jaw part or parts.

In an electrosurgical tool as described here, the flexible conductor component in the area of the mechanical transmission device is helical in particular.

The flexible conductor component can surround the mechanical transmission device helically or helically or be arranged inside the mechanical transmission device. In the case of a flexible mechanical transmission device in particular, a helical or helical design of the flexible conductor component can ensure that the flexible conductor component offers low mechanical resistance to bending of the transmission device.

An electrosurgical tool as described herein comprises, in particular, a plurality of second electrode surfaces on a second gripping surface on the second jaw part, which are electrically insulated from one another and can be connected or are connected independently of one another to an electrical power source or an electrical measuring device for measuring an impedance and/or another electrical property or characteristic.

An electrosurgical tool as described herein comprises, in particular, a further flexible conductor component having an electrically insulating substrate and a plurality of second electrode surfaces on a second surface area (second surface region) on the further flexible conductor component, wherein at least a part area of the further flexible conductor component is mechanically connected to the second jaw part, wherein the second surface area of the flexible conductor component with the plurality of second electrode surfaces forms at least a part of a second gripping surface on the second jaw part.

The further flexible conductor component has in particular features, properties and functions of a flexible conductor component of an electrosurgical tool described herein. The other flexible conductor component is also flat, i.e. thin and substantially two-dimensional. The second electrode surfaces of the plurality of second electrode surfaces are exposed at the second gripping surface at the second jaw part. This means that each individual second electrode surface of the plurality of second electrode surfaces between the gripping surfaces of the jaw parts can directly mechanically contact held or crushed tissue and form a conductive contact with them. In particular, the second electrode surfaces are electrically insulated from one another and can be connected or are connected independently of one another to an electrical power source or an electrical measuring device for measuring an impedance and/or another electrical property or parameter. The further flexible conductor component can be permanently mechanically connected to the second jaw part. Alternatively, the further flexible conductor component can be mechanically connected to the second jaw part in such a manner that it can be separated from the second jaw part in a non-destructive manner without the use of tools.

In an electrosurgical tool as described herein, the flexible conductor component further comprises, in particular, a plurality of second electrode pads on a second surface area on a second part area of the flexible conductor component, wherein the first part area of the flexible conductor component comprising the first surface area and the second part area of the flexible conductor component comprising the second surface area are mechanically connected to the first jaw part.

The first part area and the second part area of the flexible conductor component are arranged in particular parallel to one another and on both sides of a bead and/or a groove on the gripping surface of the first jaw part. The bead can be provided and configured to compress gripped fabric. The groove can be provided and configured to guide a mechanical cutting tool between the jaw parts.

In an electrosurgical tool as described herein, the flexible conductor component further comprises, in particular, a plurality of second electrode surfaces on a second surface area on a second part area of the flexible conductor component, wherein the second part area of the flexible conductor component comprising the second surface area is mechanically connected to the second jaw part, wherein the second surface area of the flexible conductor component with the plurality of second electrode surfaces forms at least part of a second gripping surface on the second jaw part.

The flexible conductor component can thus have a first part area with a plurality of first electrode surfaces, which forms the first gripping surface on the first jaw part, and a second part area with a plurality of second electrode surfaces, which forms the second gripping surface on the second jaw part. The flexibility of the conductor component allows the two jaw parts to move relative to one another.

In particular, the second electrode surfaces of the plurality of second electrode surfaces are electrically insulated from one another and can be connected or are connected independently of one another to an electrical power source or an electrical measuring device for measuring an impedance and/or another electrical property or parameter. In particular, all first electrode surfaces of the plurality of first electrode surfaces and all second electrode surfaces of the plurality of second electrode surfaces are electrically insulated from one another and can be connected or are connected independently of one another to an electrical power source or an electrical measuring device for measuring an impedance and/or another electrical property or parameter. Alternatively, a plurality of first electrode surfaces and/or second electrode surfaces can be connected in parallel within one or more groups.

The second part area of the flexible conductor component can be permanently mechanically connected to the second jaw part. This means that it cannot be non-destructively detached from the second jaw part without using a screwdriver, wrench or other tool, or cannot be non-destructively detached from the second jaw part even when a screwdriver, wrench or other tool is used. Alternatively, the second part area of the flexible conductor component can be mechanically connected to the second jaw part in such a manner that it can be separated non-destructively from the second jaw part without using a screwdriver, wrench or other tool. The mechanical connection between the flexible conductor component and the first jaw part and the mechanical connection between the second part area of the flexible conductor component and the second jaw part may be the same or different.

Forming the first gripping surface of the first jaw part and the second gripping surface of the second jaw part using the same flexible conductor component can simplify the manufacture of the electrosurgical tool and, above all, the contacting of the electrode surfaces.

In an electrosurgical tool as described here, the flexible conductor component has in particular a fork-shaped form.

In an electrosurgical tool as described herein, the flexible conductor component has, in particular, a fork-shaped configuration with the first part area at the first jaw part and the second part area at the second jaw part.

The first part area of the flexible conductor component with the first electrode surfaces on the first jaw part thus resembles a first prong, while the second part area of the flexible conductor component with the second electrode surfaces on the second jaw part resembles a second prong of a fork. An elongated portion of the flexible conductor component, which may contain conductive paths for contacting the first and second electrode surfaces and extend to a handling device, resembles the handle of a fork.

In an electrosurgical tool as described herein, in particular at least either the first electrode surfaces or the second electrode surfaces are arranged in strips and parallel to one another.

In an electrosurgical tool as described herein, in particular, the first electrode surfaces are arranged in strips and parallel to one another and the second electrode surfaces are arranged in strips and parallel to one another and orthogonal to the first electrode surfaces.

In an electrosurgical tool as described herein, in particular at least either the first electrode surfaces or the second electrode surfaces are arranged in a pattern that repeats periodically in one or more directions.

For example, the first electrode surfaces and/or the second electrode surfaces are each arranged in straight strips with constant and equal widths and parallel to one another and with equal spacing. Alternatively, the first electrode surfaces and/or the second electrode surfaces each have the shape of circles, ellipses, rectangles, hexagons or other polygons, for example, and are arranged in the form of a rectangular or oblique-angled (in particular hexagonal) grid or array that is periodic in two directions.

In an electrosurgical tool as described herein, in particular the first electrode surfaces are arranged in a first plane or substantially in a first plane and/or the second electrode surfaces are arranged in a second plane or substantially in a second plane.

An electrosurgical instrument comprises an electrosurgical tool as described herein.

An electrosurgical instrument comprises an electrosurgical tool as described herein and a handling device, which is mechanically and functionally connectable or connected to the tool, for manually or mechanically controlling functions of the electrosurgical instrument.

An electrosurgical instrument comprises a shaft having a proximal end mechanically and functionally couplable or coupled to a handling device for manually or mechanically controlling functions of the electrosurgical instrument, and a distal end and an electrosurgical tool as described herein mechanically and functionally couplable or coupled to the distal end of the shaft.

Optionally, the electrosurgical instrument further comprises a handling device that is mechanically and functionally couplable or coupled to the proximal end of the shaft.

A method of manufacturing an electrosurgical tool for an electrosurgical instrument comprises a step of providing a first jaw part, a step of providing a second jaw part, a step of providing a flexible conductor component having an electrically insulating substrate and a plurality of first electrode surfaces on a first surface area on a first portion of the flexible conductor component, and a step of mechanically connecting at least the first portion of the flexible conductor component to the first jaw part so that the first surface area with the plurality of first electrode surfaces on the first portion of the flexible conductor component forms at least part of a first gripping surface on the first jaw part.

In particular, the method is used to manufacture an electrosurgical tool as described herein, i.e. with features, properties and functions of the electrosurgical tool described herein.

An electrosurgical tool produced by means of the method has, in particular, features, properties and functions of an electrosurgical tool described herein. In particular, a flat flexible conductor component, i.e. a thin and essentially two-dimensional flexible conductor component, is provided. In this case, the electrode surfaces of the plurality of first electrode surfaces are exposed on the first gripping surface on the first jaw part. This means that each individual electrode surface of the plurality of first electrode surfaces between the gripping surfaces of the jaw parts can directly mechanically contact held or crushed tissue and form a conductive contact with it. In particular, the electrode surfaces are electrically insulated from one another and can be connected or are connected independently of one another to an electrical power source or an electrical measuring device for measuring an impedance and/or another electrical property or parameter.

The flexible conductor component can be permanently mechanically connected to the first jaw part. Alternatively, the flexible conductor component can be mechanically connected to the first jaw part in such a manner that it can be separated from the first jaw part non-destructively without the use of tools.

In particular, a method as described herein further comprises a step of providing a further flexible conductor component having an electrically insulating substrate and a plurality of second electrode surfaces on a second surface area on the further flexible conductor component, and a step of mechanically connecting at least a part area of the further flexible conductor component to the second jaw part so that the second surface area on the further flexible conductor component forms the second gripping surface on the second jaw part.

The further flexible conductor component also has, in particular, features, properties and functions of a further flexible conductor component of an electrosurgical tool described herein.

In particular, a method as described herein further comprises a step of mechanically connecting a further part area of the flexible conductor component to the second jaw part such that a second surface area having a plurality of second electrode surfaces on the second part area of the flexible conductor component forms a second gripping surface on the second jaw part.

In particular, the flexible conductor component is permanently connected to the second jaw part. This means that the flexible conductor component cannot be separated from the second jaw part non-destructively, at least without using a screwdriver, wrench or other tool. Alternatively, the flexible conductor component is connected to the second jaw part in a non-destructive, detachable manner, for example. In particular, the second electrode surfaces on the second part area of the flexible conductor component are electrically insulated from one another and can be connected independently of one another to an electrical power source or an electrical measuring device.

In the following, embodiments are explained in more detail with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of an electrosurgical instrument;

FIG. 2 is a schematic representation of an electrosurgical tool of the electrosurgical instrument shown in FIG. 1;

FIG. 3 is a schematic representation of a sectional view of the electrosurgical tool shown in FIGS. 1 and 2;

FIG. 4 is a schematic representation of a flexible circuit board of the electrosurgical tool from FIGS. 1 to 3;

FIG. 5 is a schematic flow chart of a process for manufacturing an electrosurgical tool.

### DESCRIPTION OF EMBODIMENTS

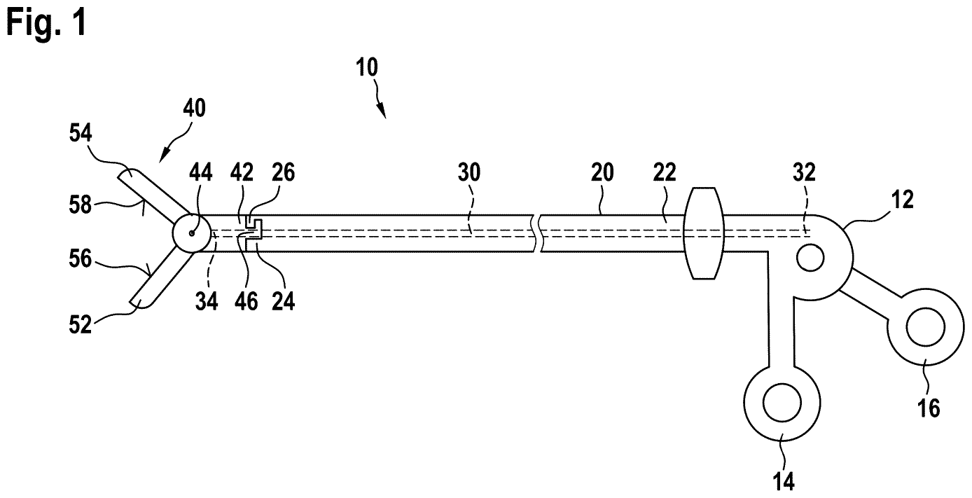

Referring to the drawings, FIG. 1 shows a schematic representation of an electrosurgical instrument 10. A handling device 12 forms a proximal end of the electrosurgical instrument 10. The handling device 12 has a fixed handle part 14 and a handle part 16 which can be moved relative to the fixed handle part 14, for example pivoted about a pivot axis orthogonal to the drawing plane of FIG. 1.

The electrosurgical instrument 10 further comprises a shaft 20. A proximal end 22 of the shaft 20 is mechanically connected to the handling device 12. The mechanical connection between the proximal end 22 of the shaft 20 and the handling device 12 can be permanently or non-destructively detachable.

In the example shown, the distal end 24 of the shaft 20 is formed by a coupling device 26.

The electrosurgical instrument 10 further comprises a transmission device 30 arranged in the shaft 20. The transmission device 30 has a proximal end 32 and a distal end 34. The transmission device 30 is provided and configured to transmit a translational movement and a corresponding force and/or a rotational movement and a corresponding torque between its proximal end 32 and its distal end 34.

The electrosurgical instrument 10 further comprises an electrosurgical tool 40. A proximal end 42 of the electrosurgical tool 40 is mechanically connected to the distal end 24 of the shaft 20 in a permanent or non-destructive detachable manner. Furthermore, the electrosurgical tool 40 comprises a joint 44.

In the example shown, a coupling device 46 corresponding to the coupling device 26 at the distal end 24 of the shaft 20 forms the proximal end 42 of the electrosurgical tool 40. The coupling device 26 at the distal end 24 of the shaft 20 and the corresponding coupling device 46 at the proximal end 42 of the electrosurgical tool 40 form a particularly rigid but detachable mechanical connection between the electrosurgical tool 40 and the shaft 20.

The electrosurgical tool 40 comprises a first jaw part 52 and a second jaw part 54. In the example shown, both jaw parts 52, 54 are mechanically connected to the proximal end 42, namely the coupling device 46, in an articulated manner by the joint 44. The joint 44 defines a pivot axis orthogonal to the drawing plane of FIG. 1, about which both jaw parts 52, 54 are pivotable.

Alternatively, and in deviation from the illustration in FIG. 1, one jaw part 52 can be rigidly connected to the proximal end 42 and the coupling device 46 and only the other jaw part 54 can be pivotably connected. Furthermore, the electrosurgical tool 40 may have more than two jaw parts 52, 54, in deviation from the embodiment in FIG. 1.

The proximal end 32 of the transmission device 30 is mechanically coupled to the movable handle part 16 of the handling device 12. The distal end 34 of the transmission device 30 is mechanically coupled to the jaw parts 52, 54 of the electrosurgical tool 40 in a manner not shown in FIG. 1. Therefore, each movement of the movable handle part 16 of the handling device 12 is accompanied by a movement of the jaw parts 52, 54. A force generated manually or by a machine on the movable handle part 16 is transmitted to the jaw parts 52, 54.

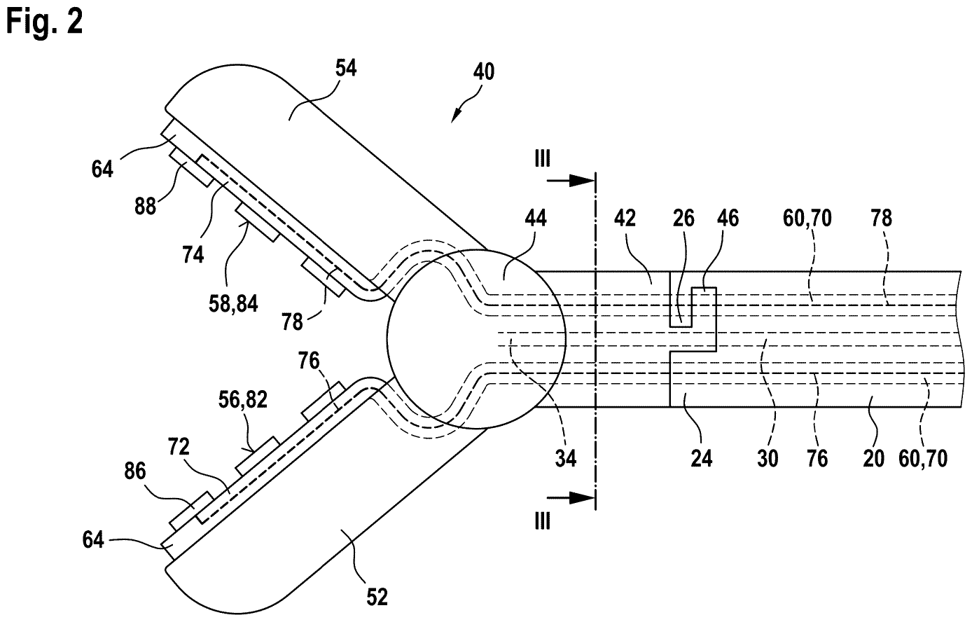

FIG. 2 shows a schematic and enlarged representation of the electrosurgical tool 40 of the electrosurgical instrument 10 of FIG. 1. The drawing plane of FIG. 2 corresponds to the drawing plane of FIG. 1.

The first jaw part 52 has a first gripping surface 56, which faces the second jaw part 54. The second jaw part 54 has a second gripping surface 58, which faces the first jaw part 52. Both gripping surfaces 56, 58 are flat, orthogonal to the drawing plane of FIG. 2 and are formed in such a manner that they can lie flat or substantially flat against one another. Alternatively and in deviation from the illustration in FIG. 2, the gripping surfaces 56, 58 can be curved (Gaussian curvature k=0) or curved (Gaussian curvature k≠0).

The electrosurgical tool 40 comprises a flexible circuit board 60 having an electrically insulating substrate 64, which may comprise a stack of laminated thin plastic films. The flexible printed circuit board 60 is thin and flat and, in particular, is initially produced in a flat shape and then permanently or detachably mechanically connected to the electrosurgical tool. The flexible printed circuit board 60 has a fork-shaped topology with a narrow strip-shaped proximal part area 70, a first distal part area 72 and a second distal part area 74. The two distal part areas 72, 74 are arranged relative to the proximal part area 70 like the prongs of a fork relative to its handle.

First conductive tracks 76 and second conductive tracks 78 are arranged in the electrically insulating substrate 64 of the flexible printed circuit board 60, in particular between the films in the case of a substrate 64 formed from a plurality of laminated films. The first conductive paths 76 extend from the first distal part area 72 to a proximal end of the proximal part area 70. The second conductive paths 78 extend from the second distal part area 74 to a proximal end of the proximal part area 70.

The first distal part area 72 of the flexible printed circuit board 60 is arranged at the first jaw part 52. At least a part of the first distal part area 72 of the flexible printed circuit board 60 is permanently or detachably mechanically connected to the first jaw part 52, for example by means of an adhesive bond or a hook-and-loop connection or form-fit. A first surface area 82 at the first distal part area 72 forms the first gripping surface 56 at the first jaw part 52 or—deviating from the representation in FIG. 2—at least a part of the first gripping surface 56 at the first jaw part 52

The second distal part area 74 of the flexible printed circuit board 60 is arranged at the second jaw part 54. At least a part of the second distal part area 74 of the flexible printed circuit board 60 is permanently or detachably mechanically connected to the second jaw part 52, for example by means of an adhesive bond or a hook-and-loop connection or form-fit. A second surface area 84 at the second distal part area 74 forms the second gripping surface 58 at the second jaw part 54 or—deviating from the representation in FIG. 2—at least a part of the second gripping surface 58 at the second jaw part 54.

The first surface area 82 at the first distal part area 72 of the flexible printed circuit board 60 has a plurality of exposed, i.e. open, first electrode surfaces 86. The first electrode surfaces 86 thus form parts of the first gripping surface 56 on the first jaw part 52. The first electrode areas 86 may be formed by conductive structures—in particular exposed areas of first conductive tracks 76—on the surface of the electrically insulating substrate 64.

As indicated in FIG. 2, the first electrode surfaces 86 may be raised relative to the regions of the first gripping surface 56 formed by the surface of the electrically conductive substrate 64. Alternatively, and in contrast to the embodiment in FIG. 2, the electrically conductive structures forming the first electrode surfaces 86 may be partially or completely embedded in the electrically conductive substrate 64. In this case, the first electrode surfaces 86 are less protruding towards the second jaw part 54 with respect to the areas of the first gripping surface 56 formed by the surface of the electrically conductive substrate 64 or are flush with these areas.

Each first electrode surface 86 is electrically conductively connected to a distal end of a first conductive track 76. In particular, each first electrode surface 86 is formed on a first conductive track 76 or on an electrically conductive structure formed integrally or monolithically with and/or merging into a first conductive track 76. Each first conductive track 76 can be electrically conductively connected to a first electrode surface 86 or to a plurality of first electrode surfaces 86 (thus electrically connected in parallel).

The second surface area 84 at the second distal part area 74 of the flexible printed circuit board 60 has a plurality of exposed, i.e. open, second electrode surfaces 88. The second electrode surfaces 88 thus form parts of the second gripping surface 58 on the second jaw part 54. The second electrode areas 88 may be formed by conductive structures—in particular exposed areas of second conductive tracks 78—on the surface of the electrically insulating substrate 64.

As indicated in FIG. 2, the second electrode surfaces 88 may be raised relative to the regions of the second gripping surface 58 formed by the surface of the electrically conductive substrate 64. Alternatively, and in contrast to the embodiment in FIG. 2, the electrically conductive structures forming the second electrode surfaces 88 may be partially or completely embedded in the electrically conductive substrate 64. In this case, the second electrode surfaces 88 are less protruding towards the first jaw part 52 with respect to the areas of the second gripping surface 58 formed by the surface of the electrically conductive substrate 64 or are flush with these areas.

Each second electrode surface 88 is electrically conductively connected to a distal end of a second conductive track 78. In particular, each second electrode surface 88 is formed on a second conductive track 78 or on an electrically conductive structure formed integrally or monolithically with and/or merging into a second conductive track 78. Each second conductive track 78 can be electrically conductively connected to a second electrode surface 88 or to a plurality of second electrode surfaces 88 (thus electrically connected in parallel).

In the area of the joint 44 of the electrosurgical jaw part 40 and proximal thereto, the distal part areas 72, 74 and the proximal part area 70 of the flexible circuit board 60 are hidden inside the joint 44 and inside the shaft 20 and thus not visible from the outside. Therefore, the distal part areas 72, 74 and the proximal part area 70 of the flexible circuit board 60 inside the joint 44 and inside the shaft 20 are shown by dashed lines.

In the example shown, the distal part areas 72, 74 of the flexible circuit board 60 in the area of the joint 44 of the electrosurgical jaw part 40 are bent to provide increased elasticity. This increased elasticity facilitates the pivoting of the jaw parts 52, 54 about the pivot axis defined by the joint 44 and the necessary bending of the distal part areas 72, 74 of the flexible printed circuit board 60. Furthermore, this increased elasticity allows the changes in those distances bridged by the distal part areas 72, 74 caused by the pivoting.

The proximal part area 70 of the flexible printed circuit board 60 is arranged in the shaft 20. In the example shown, the proximal part area 70 of the flexible printed circuit board 60 is mechanically rigidly connected to the transmission device 30 and partially surrounds it in the circumferential direction.

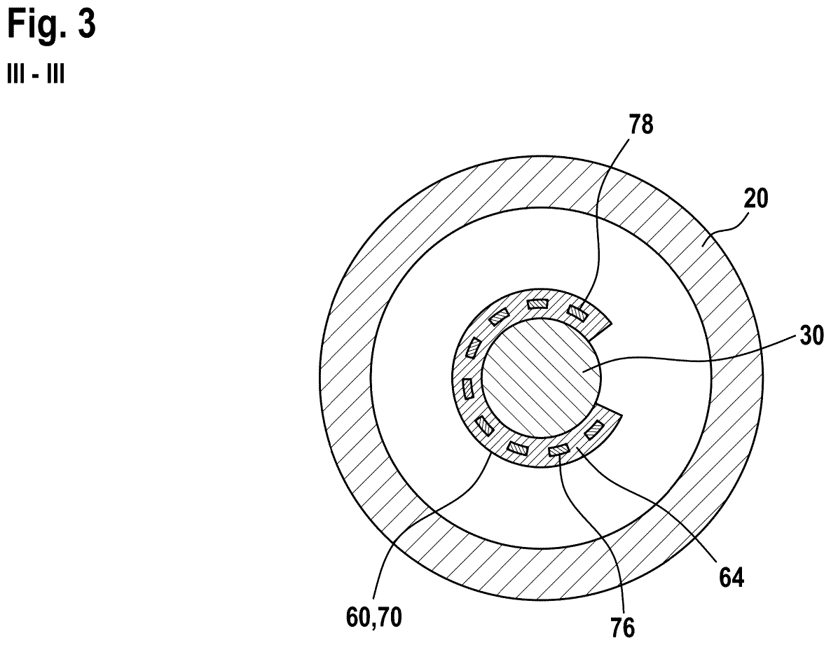

FIG. 3 shows a schematic representation of a section through the electrosurgical tool 40 from FIGS. 1 and 2. The sectional plane is orthogonal to the drawing planes of FIGS. 1 and 2 and lies between the joint 44 and the proximal end 42 of the electrosurgical tool 40 formed by the coupling device 46. The position of sectional plane III-III in FIG. 3 is indicated in FIG. 2. A cut through the shaft 20 would look similar.

In the example shown, the transmission device 30 is rod-shaped with a circular cross-section. The proximal part area 70 of the flexible printed circuit board 60 largely surrounds the transmission device 30 in the circumferential direction, i.e. has a C-shaped cross-section. The proximal part area 70 of the flexible printed circuit board 60 is connected to the outer surface of the transmission device 30, in particular in a planar manner, for example by means of an adhesive bond. The flexible printed circuit board 60 can be arranged parallel to the transmission device 30 or surround it helically in order to offer only slight resistance to bending of the transmission device 30.

The first conductive tracks 76 and the second conductive tracks 78 are arranged in the electrically conductive substrate 64. The conductive paths 76, 78 are electrically insulated from each other and from the environment, in particular from the shaft 20 and from the transmission device 30, by the electrically insulating substrate 64.

Alternatively, and in contrast to the embodiment shown in FIGS. 2 and 3, the proximal part area 70 of the flexible printed circuit board 60 may not be mechanically connected to the transmission device 30, but to the inner surface of the tube forming the shaft 20. Furthermore, the proximal part area 70 of the flexible printed circuit board 60 may be mechanically connected neither to the transmission device 30 nor to the tube forming the shaft 20.

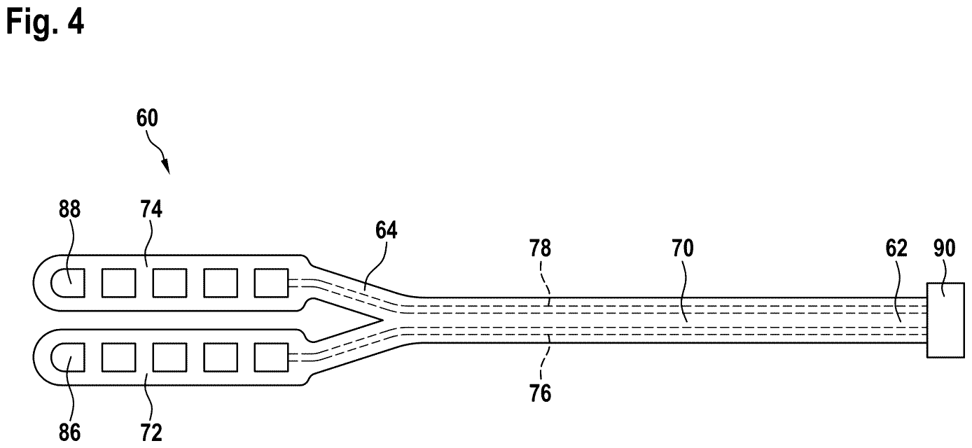

FIG. 4 shows a schematic top view of the flexible printed circuit board 60 of the electrosurgical tool 40 of FIGS. 1 to 3. FIG. 4 shows the flexible printed circuit board 60 in a flat shape, in particular as it is immediately after the production of the flexible printed circuit board 60. The drawing plane of FIG. 4 is parallel to the two large and, in the configuration shown in FIG. 4, flat surface areas of the flexible printed circuit board 60. The electrically insulating substrate 64 of the flexible printed circuit board 60 can be optically transparent or opaque or largely or completely opaque. Therefore, the contours of the first and second conductive tracks 76, 78 inside the electrically insulating substrate 64 are indicated by dashed lines.

The flexible printed circuit board 60 has a fork-shaped topology in which the narrow strip-shaped proximal part area 70 resembles the handle and the distal part areas 72, 74 resemble the prongs of a fork.

At the proximal end 62 of the proximal part area 70 of the flexible printed circuit board 60, a multipole connector 90 is provided for the detachable mechanical and electrical connection of the flexible printed circuit board 60 and its conductive tracks 76, 78 to a corresponding connector in or on the handling device 12 (see FIG. 1) or to a cable. Impedances and/or other electrical properties or characteristics between pairs of electrode surfaces 86, 88 or other electrical measured values can be detected, or electrical voltages can be applied to pairs of electrode surfaces 86, 88 via the connector 90.

Alternatively, and in deviation from the embodiment with reference to FIGS. 1 to 4, the flexible printed circuit board 60 can have only a (in particular strip-shaped) distal part area, which forms only the first gripping surface 56 of the first jaw part 52. This first jaw part 52 may be rigidly or movably connected to the proximal end 42 of the electrosurgical tool 40 by a joint 44. The second gripping surface 58 of the second jaw part 54 may have second electrode surfaces 88, but these are not formed by the flexible printed circuit board 60. For example, the second electrode surfaces 88 may be formed on a distal portion of another flexible printed circuit board that forms the second gripping surface 58 on the second jaw part 54.

In particular, the transmission device 30 is hingedly but permanently mechanically connected to the electrosurgical tool 40, namely to its jaw parts 52, 54. After releasing the mechanical connection between the coupling device 26 at the distal end 24 of the shaft 20 and the corresponding coupling device 46 at the proximal end 42 of the electrosurgical tool 40, the transmission device 30 can be pulled distally out of the tube forming the shaft 20. The same applies to the proximal part area 70 of the flexible printed circuit board.

Alternatively, and in contrast to the embodiment shown in FIGS. 1 to 4, the electrosurgical tool 40 may be permanently mechanically connected to the distal end 24 of the shaft 20.

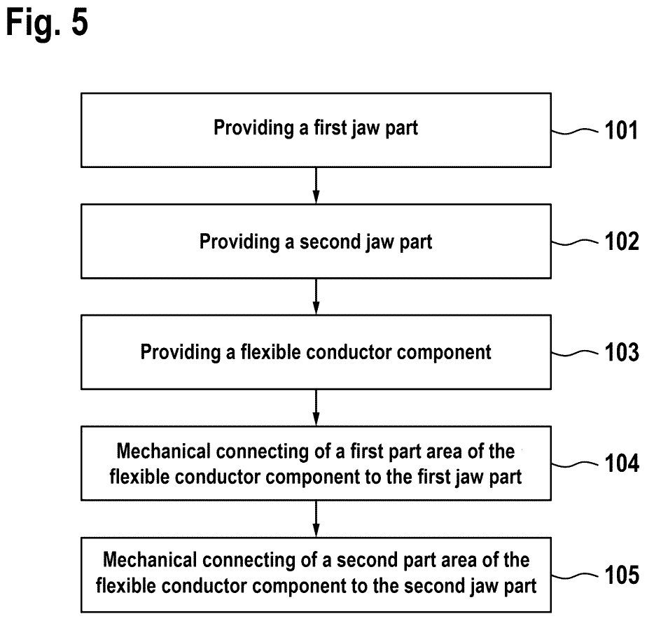

FIG. 5 shows a schematic flow chart of a process for manufacturing an electrosurgical tool. The method is particularly suitable for manufacturing the electrosurgical tool 40 shown in FIGS. 1 to 3. However, the method is also suitable for manufacturing an electrosurgical tool with features, properties and functions that differ from those shown in FIGS. 1 to 4. Nevertheless, reference symbols from FIGS. 1 to 3 are used as examples.

At a first step 101, a first jaw part 52 is provided. At a second step 102, a second jaw part 54 is provided. At a third step 103, a flexible conductor component, in particular a flexible printed circuit board 60 having a proximal part area 70, one or more (in particular distal) part areas 72, 74, first electrode areas 86 on a first surface area 82 on the first part area 72 and optionally second electrode areas 88 on a second surface area 84 on a second distal part area 74 is provided.

At a fourth step 104, the first part area 72 of the flexible conductor component 60 is permanently or releasably mechanically connected to the first jaw part 52 such that the first surface area 82 on the first part area 72 of the flexible conductor component 60 forms the first gripping surface 56 on the first jaw part 52.

At an optional fifth step 105, the second part area 74 of the flexible conductor component 60 is permanently or releasably mechanically connected to the second jaw part 54 such that the second surface area 84 on the second part area 74 of the flexible conductor component 60 forms the second gripping surface 58 on the second jaw part 54.

While specific embodiments of the invention have been shown and described in detail to illustrate the application of the principles of the invention, it will be understood that the invention may be embodied otherwise without departing from such principles.

### REFERENCE NUMBERS

10 electrosurgical instrument
12 handling device of the electrosurgical instrument 10
14 fixed handle part of the handling device 12
16 movable handle part of the handling device 12
20 shaft of the electrosurgical instrument 10
22 proximal end of the shaft 20
24 distal end of the shaft 20
26 coupling device at the distal end 24 of the shaft 20, for releasable mechanical connection with the electrosurgical tool 40
30 transmission device in the shaft 16
32 proximal end of the transmission device 30
34 distal end of the transmission device 30
40 electrosurgical tool of the electrosurgical instrument 10
42 proximal end of the electrosurgical tool 40
44 joint of the electrosurgical tool 40
46 coupling device at the proximal end 42 of the electrosurgical tool 40
52 first jaw part of the electrosurgical tool 40
54 second jaw part of the electrosurgical tool 40
56 first gripping surface on the first jaw part 52
58 second gripping surface on the second jaw part 54
60 flexible circuit board of the electrosurgical tool 40
62 proximal end of the flexible circuit board 60
64 electrically insulating substrate of the flexible printed circuit board 60
66 electrical connector at the proximal end 62 of the flexible printed circuit board 60
70 proximal part area of the flexible printed circuit board 60
72 first distal part area of the flexible printed circuit board 60
74 second distal part area of the flexible printed circuit board 60
76 first conductive tracks in the flexible printed circuit board 60
78 second conductive tracks in the flexible printed circuit board 60
82 first surface area on the first distal part area 72 of the flexible printed circuit board 60
84 second surface area on the second distal part area 74 of the flexible printed circuit board 60
86 first electrode surface on the first surface area 76 of the flexible printed circuit board 60
88 second electrode surface on the second surface area 78 of the flexible printed circuit board 60
90 multipole plug connector
101 first step (providing a first jaw part)
102 second step (providing a second jaw part)
103 third step (providing a flexible conductor component)
104 fourth step (mechanical connection of a first part area of the flexible conductor component to the first jaw part)
105 fifth step (mechanical connection of a second part area of the flexible conductor component to the second jaw part)

1. An electrosurgical tool for an electrosurgical instrument, the electrosurgical tool comprising:
a first jaw part;
a second jaw part;
a flexible conductor component having an electrically insulating substrate and a plurality of first electrode surfaces on a first surface area of the flexible conductor component,
wherein at least a first part area of the flexible conductor component is mechanically connected to the first jaw part,
wherein the first surface area of the flexible conductor component with the plurality of first electrode surfaces forms at least part of a first gripping surface on the first jaw part.

2. The electrosurgical tool according to claim 1, wherein the flexible conductor component comprises a plurality of conductive tracks, and each of the plurality of conductive tracks is electrically conductively connected to one of the plurality of first electrode surfaces.

3. The electrosurgical tool according to claim 1, further comprising: a joint hingedly connecting at least either the first jaw part or the second jaw part to a distal end of a shaft or to a mechanical coupling device or to a handling device, wherein the flexible conductor component extends from the first jaw part over the joint to a location proximal to the joint.

4. The electrosurgical tool according to the preceding claim, wherein the flexible conductor component extends from the first jaw part through the joint or past the joint to a handling device.

5. The electrosurgical tool according to claim 1, wherein the flexible conductor component extends from the first jaw part via the joint to a proximal end of a shaft, the distal end of which is mechanically connected to the electrosurgical tool.

6. The electrosurgical tool according to claim 1, further comprising: an electrical connector mechanically and electrically connected to a proximal end of the flexible conductor component.

7. The electrosurgical tool according to claim 1, further comprising: a mechanical transmission device for transmitting at least either a manually or mechanically generated force or a manually or mechanically generated torque to the electrosurgical tool, for moving at least either the first jaw part or the second jaw part, wherein the flexible conductor component is integrated or mechanically rigidly connected to the transmission device.

8. The electrosurgical tool according to claim 7, wherein the flexible conductor component is helical in an area of the mechanical transmission device.

9. The electrosurgical tool according to claim 1, wherein the flexible conductor component further comprises a plurality of second electrode surfaces at a second surface area at a second part area of the flexible conductor component, wherein the second part area of the flexible conductor component, which comprises the second surface area, is mechanically connected to the second jaw part, and the second surface area of the flexible conductor component with the plurality of second electrode surfaces forms at least part of a second gripping surface on the second jaw part.

10. The electrosurgical tool according to claim 1, wherein the flexible conductor component has a bifurcated configuration with the first part area at the first jaw part and the second part area at the second jaw part.

11. The electrosurgical tool according to claim 1, in combination with a shaft with a proximal end configured to be mechanically and functionally coupled or coupled to a handling device for manual or mechanical control of functions of the electrosurgical instrument, and a distal end, wherein the electrosurgical tool is mechanically and functionally couplable or coupled to the distal end of the shaft.

12. A method of manufacturing an electrosurgical tool for an electrosurgical instrument, the method comprising the following steps:
providing a first jaw part;
providing a second jaw part;
providing a flexible conductor component having an electrically insulating substrate and a plurality of first electrode surfaces at a first surface area at a first part area of the flexible conductor component;
mechanically connecting at least the first part area of the flexible conductor component to the first jaw part to form at least a part of the first gripping surface, such that the first surface area of the flexible conductor component with the plurality of first electrode surfaces forms at least a part of a first gripping surface on the first jaw part.

13. The method according to claim 12, further comprising the following step: mechanically connecting a second part area of the flexible conductor component to the second jaw part so that a second surface area with a plurality of second electrode surfaces on the second part area of the flexible conductor component forms a second gripping surface on the second jaw part.
